(19) United States
(12) Patent Application Publication
(10) Pub. No.: US 2007/0078513 A1
(43) Pub. Date: Apr. 5, 2007

(54) Controllable drug releasing gradient coatings for medical devices

(75) Inventor: Todd Campbell, Petaluma, CA (US)

Correspondence Address: MEDTRONIC VASCULAR, INC.;IP LEGAL DEPARTMENT, 3576 UNOCAL PLACE, SANTA ROSA, CA, 95403, US

(73) Assignee: MEDTRONIC VASCULAR, INC., SANTA ROSA, CA (US)

(21) Appl. No.: 10/527,414

(22) PCT Filed: Sep. 18, 2003
(86) PCT No.: PCT/US03/30010

§ 371 Date: Nov. 9, 2006

Related U.S. Application Data
(60) Provisional application No. 60/411,852, filed on Sep. 18, 2002.

(51) Int. Cl. A61F 2/06; A61F 2/00

(52) U.S. Cl. 623/1.44; 623/901; 424/426

(57) ABSTRACT

Implantable medical devices having a polymer gradient coating capable of controllably releasing at least one pharmaceutical compound to a localized area are disclosed. More specifically, the gradient coatings comprise at least two layers where at least one of these layers incorporates at least one pharmaceutical compound. Each of the layers of the gradient coating has at least one physical property affecting the releasability of the pharmaceutical compound incorporated therein that differs from that of at least one other layer. These physical properties include, but are not limited to, solubility constants, molecular weights, elution profiles, and bonding strengths.

### FIELD OF INVENTION

The present invention generally relates to medical devices and to methods of making and using medical devices to controllably deliver pharmaceutical compounds to specific locations within a patients body. More specifically, the present invention is directed to drug releasing coatings for medical devices that provide relatively precise control of the timing, quantities, and types of pharmaceutical compounds released from the coatings following implantation of the medical devices.

### BACKGROUND OF THE INVENTION

The implantation of medical devices has become a relatively common technique for treating a variety of medical or disease conditions within a patient's body. Depending upon the conditions being treated, today's medical implants can be positioned within specific portions of a patient's body where they can provide beneficial functions for periods of time ranging from days to years. A wide variety of medical devices can be considered implants for purposes of the present invention. Such medical devices can include structural implants such as stents and internal scaffolding for vascular use, replacement parts such as vascular grafts, or in-dwelling devices such as probes, catheters and microparticles for monitoring, measuring and modifying biological activities within a patient's cardiovascular system. Other types of medical implants for treating different types of medical or disease conditions can include in-dwelling access devices or ports, valves, plates, barriers, supports, shunts, discs, and joints, to name a few.

For example, cardiovascular disease, commonly referred to as atherosclerosis, remains a leading cause of death in developed countries. Atherosclerosis is a disease that results in the narrowing, or stenosis, of blood vessels which can lead to heart attack or stroke if the narrowing progresses to the point of blocking blood flow through the narrowed blood vessels forming the coronary arteries. Cardiovascular disease caused by stenotic or narrowed coronary arteries is commonly treated using either a coronary artery by-pass graft (CABG) around the blockage, or a procedure called angioplasty where a balloon catheter is inserted into the blocked coronary artery and advanced until the vascular stenosis is reached by the advancing balloon. The balloon is then inflated to deform the stenosis open, restoring blood flow.

However, angioplasty or balloon catheterization can result in internal vascular injury which may ultimately lead to reformation of narrowing vascular deposits within the previously opened artery. This biological process whereby a previously opened artery becomes re-occluded is referred to as restenosis. One angioplasty variation designed to reduce the possibility of restenosis includes the subsequent step of arterial stent deployment within the stenotic blockage opened by the expanded balloon. After arterial patency has been restored by expanding the angioplasty balloon to deform the stenotic lesion open, the balloon is deflated and a vascular stent is inserted into the tubular bore or vessel lumen across the stenosis site. The catheter is then removed from the coronary artery lumen and the deployed stent remains implanted across the opened stenosis to prevent the newly opened artery from constricting spontaneously or narrowing in response to the internal vascular injury resulting from the angioplasty procedure itself. However, it has been found that in some cases of angioplasty and angioplasty followed by stent deployment that restenosis may still occur.

Treating restenosis generally requires additional, more invasive, procedures including CABG in some cases. Consequently, methods for preventing restenosis, or for treating incipient forms of restenosis, are being aggressively pursued. One promising method for preventing restenosis is the administration of medicaments that block the local invasion or activation of monocytes, white blood cells that respond to injury or infection, thus preventing the associated secretion of growth factors within the blood vessel at the restenosis site that can trigger vascular smooth muscle cell (VSMC) proliferation and migration causing thickening of the vessel wall and subsequent narrowing of the artery. Metabolic inhibitors such as anti-neoplastic agents are currently being investigated as potential anti-restenotic compounds for such purposes. However, the toxicity associated with the systemic administration of known metabolic inhibitors has more recently stimulated development of in situ or site-specific drug delivery designed to place the anti-restenotic compounds directly at the target site within the potential restenotic lesion rather than generally administering much larger, potentially toxic doses to the patient.

For example, one particular site-specific drug delivery technique known in the art employs the use of vascular stents coated with anti-restenotic drugs. These stents have been particularly useful because they not only provide the mechanical structure to maintain the patency or openness of the damaged vessel, but they also release the anti-restenotic agents directly into the surrounding tissue. This site specific delivery allows clinically effective drug concentrations to be achieved locally at the stenotic site without subjecting the patient to the side effects that may be associated with systemic drug delivery of such pharmaceutical compounds. Moreover, localized or site specific delivery of anti-restenotic drugs eliminates the need for more complex specific cell targeting technologies intended to accomplish similar purposes.

An important factor in the efficacy of in situ drug delivery is how the drug is attached to the stent and delivered to the target site as a result. More specifically, a sufficient amount of deliverable drug needs to be releasably attached to and associated with the stent or implantable drug delivery vehicle. Typically, as known in the art, anti-restenotic drugs are releasably attached to the surfaces of implantable drug delivery devices such as stents through chemical bonding with the surface through either non-covalent or covalent bonding. Non-covalent bonds are generally weaker than covalent chemical bonds and therefore release the bound drugs more easily. Conversely, covalent chemical bonds are generally stronger and hold on to the bound drugs more securely, providing easier handling and storage.

An alternative approach to binding pharmaceutical compounds to the surfaces of implantable medical devices utilizes coatings rather than binding the drugs directly to the surfaces of the implants. For example, drugs can be incorporated into or applied to a polymer layer that is itself applied to the surface of the implant. A variety of polymers have been developed in the art which are intended to allow for drug attachment to medical implants and for subsequent delivery such those materials disclosed in U. S. Pat. Nos. 6,278,018, 6,214,901, and 5,858,653, incorporated herein by reference.

As noted above, an important factor in the efficacy and the utility of such in situ drug delivery techniques and devices is the ability to release an effective dose of the drug at the appropriate time for the appropriate duration. In most prior art technologies the drug delivering implants are coated with a polymer that binds or holds the drug within the polymer coating and releases the drug as the polymer coating is broken down by normal processes within the patients body or the drug simply diffuses out of the polymer coating once it is in an aqueous or wet environment Typically, these drug release mechanisms result in what is known as dumping, or the relatively sudden release of the majority of the bound drugs over a relatively short period of time as shown in the exemplary prior art drug release profile graphically illustrated in FIG. 1.

As shown in FIG. 1, the bulk of the releasably bound drug or drugs associated with the coated implants is released shortly after implantation. Additionally, this sudden release profile results in the amount of drug being delivered to the target site rapidly tapering off over time. As a result, an effective drug dose is delivered only for a short period of time following implantation. This can result in a less than effective administration of the drug. Thus, while these prior art drug releasing coating technologies have been useful and promising, a strong need exists for a site specific drug delivery technology utilizing medical implants where the drug release profiles and the associated drug dosages can be controlled over time. It is an object of the present invention to address this and other needs.

### SUMMARY OF THE INVENTION

Accordingly, the present invention provides controllable drug releasing medical coatings, controllable drug releasing coated medical implants, and methods for their manufacture and use. The release profile of one or more pharmaceutical compounds releasably bound to the coatings of the present invention can be controlled to provide more appropriate and desirable time released targeted in situ drug delivery of effective amounts of the pharmaceutical compounds. In a broad aspect, these and other objectives are achieved by the present invention through gradient coatings and combinations thereof having layered variations in physical properties such as solubility constants, molecular weights, elution profiles, and bonding strengths designed in a pattern to provide a desired drug release profile. These gradient coatings can be formed of polymeric materials having a wide variety of physical properties including drug retention and releasable drug bonding, though other materials including dissolvable organic and ionic compounds are also contemplated as being within the scope of the present invention.

In one exemplary embodiment of the present invention, the gradient coating is formed through the simple process of the sequential layering of two or more differing molecular weight polymers upon the surface of an implant. In this embodiment the highest molecular weight polymer is closest to the surface of the implant while the lowest molecular weight polymer is farthest from the implant. Because the degradation of the polymers is a function of their respective molecular weights, the lower molecular weight outer layer provides for the initial release of one or more pharmaceutical compounds that may be bound therein, while the heavier molecular weight polymer layer underneath provides for the slower and more prolonged release of any pharmaceutical compounds contained therein after the relatively lighter molecular weight outer layer has degraded and exposed the heavier molecular weight layer underneath.

Those skilled in the art will appreciate that additional layers may be incorporated between these two layers to provide further variations in drug release profiles. Further, the gradient need not be from heavier molecular weight to lighter, but may be the converse or even non-linear gradients. What is more, the gradient need not be limited to variations in molecular weights but can be based upon a wide variety of properties including dissolution profiles, binding strengths, solubility, and any other physical properties that may affect the quantity, rate, and duration of drug delivery. Moreover, the layers need not be limited to polymers alone and can include gradients formed of different types of physically compatible materials. Also, not every layer in the gradients of the present invention need be provided with a pharmaceutical compound or compounds releasably bound therein. Gradient layers including empty or blank layers are contemplated as being within the scope of the present invention, as are layers having differing mechanisms of drug release.

Alternative mechanisms of varying the release profile of one or more of the gradient layers of the present invention are also contemplated as being within the scope thereof. These include the utilization of ionizing radiation or pre-hydrolysation to affect the molecular weight of one or more of the gradient layers.

Similarly, it is also contemplated as being within the scope of the present invention to provide gradient layers containing differing quantities or types of pharmaceutical compounds. In this manner, it is possible to produce gradient coatings that will release one or more drugs in different quantities and at different times throughout the release profile of the gradient coating.

The controllable drug releasing gradient coatings of the present invention can be applied to a wide variety of medical implants including, but not limited to, stents, catheters, micro-particles, probes, and vascular grafts, as well as virtually any device intended to spend time within a patients body or vasculature. Depending upon the type of materials used to form the gradient coatings of the present invention, the coatings can be applied to the surface of a medical device through any of the coating processes known or developed in the art.

In accordance with the teachings of the present invention, the properties of the gradient coatings can be designed to provide a drug release profile that is appropriate for the pharmaceutical compound or compounds in use as well as for the intended target site addressed by the gradient coated implant. For example, those skilled in the art will appreciate that simple antibiotics or steroidal compounds can be layered into a gradient coating of the present invention to provide a large initial dose of drug followed by consistent, smaller maintenance dosages to achieve the desired medical effect. Once implanted at the target site the gradient coating will begin releasing the drug as intended to the specific tissues at the target site to provide a large initial dose followed by tapering smaller dosages. Similarly, anti-restenotic compounds may be controllably delivered in the appropriate concentration to a target site over a longer period of time to prevent vessel occlusion by coating a stent with a controllable drug releasing gradient coating of the present invention containing the anti-restenotic compound or compounds appropriately dosed into the layers of the gradient coating.

### BRIEF DESCRIPTION OF THE DRAWINGS

FIG. 1 graphically illustrates the drug releasing diffusion pattern of prior art drug delivery devices;

FIG. 2 is a cross-sectional view of an exemplary medical device having a controllable drug releasing gradient coating applied on at least one of its surfaces in accordance with the teachings of the present invention;

FIG. 3 graphically illustrates the drug delivery profile of an exemplary embodiment of the present invention; and

FIG. 4 graphically illustrates the drug delivery profile of an alterative exemplary embodiment of the present invention.

### DETAILED DESCRIPTION OF THE INVENTION

The present invention provides controllable drug releasing medical coatings, controllable drug releasing coated medical implants, and methods for their manufacture and use where the release profile of one or more pharmaceutical compounds releasably bound to the implants can be controlled to provide more appropriate and desirable time released in situ drug delivery of effective amounts of the one or more pharmaceutical compounds.

In one exemplary embodiment of the present invention, the controllable drug releasing coating comprises two or more sequential layers provided on the surface of a medical device where the layers have different physical properties and at least one releasable pharmaceutical compound that is incorporated with at least one of the layers of the coating. Because the pharmaceutical compounds are incorporated with the coating layers, the release of these compounds is dependent upon the degradation rate of the coating layers. The degradation rate of the coating layers can be manipulated by changing the physical properties of the coating layer. That is, if the coating is more robust, it will take longer for the coating to degrade and delay the release of associated pharmaceutical compounds. Conversely, the release rate of the pharmaceutical compounds can be released more rapidly with weaker coating layers. In a broad aspect of the present invention, the degradation rate of the coating layers can be adjusted by varying the solubility constants, molecular weights, elution profiles, and bonding strengths of each coating layer.

According to the teachings of the present invention, the controllable drug releasing coating can be formed from a plurality of polymeric materials depending on the desired drug releasing profile. The polymeric materials can be either synthetic or natural bioabsorbable polymers. Synthetic bioabsorbable polymeric materials that can be used to form the coating layers include poly (L-lactic acid), polycaprolactone, poly(lactidecglycolide), poly(ethylene-vinyl acetate), poly(hydroxybutyrate-co-valerate), polydioxanone, polyorthoester, polyanhydride, poly(glycolic acid), poly(D,L-lactic acid), poly(glycolic acid-co-trimethylene carbonate), polyphosphoester, polyphosphoester urethane, poly(amino acids), cyanoacrylates, poly(trimethylene carbonate), poly(iminocarbonate), copoly(ether-esters) such as PEO/PLA, polyalkylene oxalates, and polyphosphazenes. According to another exemplary embodiment of the present invention, the polymeric materials can be natural bioabsorbable polymers such as, but not limited to, fibrin, fibrinogen, cellulose, starch, collagen, and hyaluronic acid.

As those skilled in the art will appreciate, these polymeric materials have inherent degradation rates when exposed to physical stresses or chemical agents. For instance, one such physical stress is the exposure of the polymeric material to fluids. More specifically, a polymeric material may degrade faster if it is exposed to a flowing fluid (such as blood flowing through a blood vessel) rather than being immersed in a fluid. Additionally, exposure to various chemical agents, such as enzymes can also effect the degradation rate of the polymeric material. That is, depending on the composition of a particular polymer, it may be susceptible to degradation by chemicals, compounds, or enzymes found within the human body.

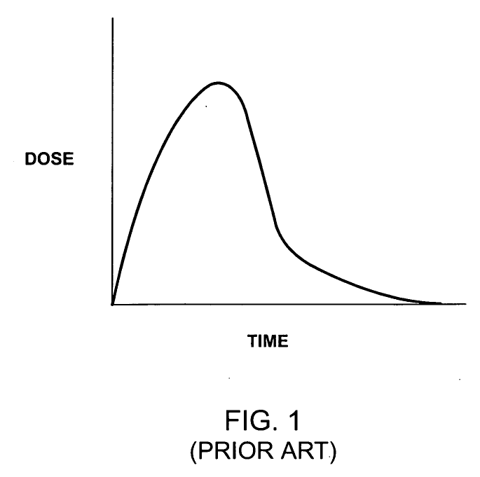

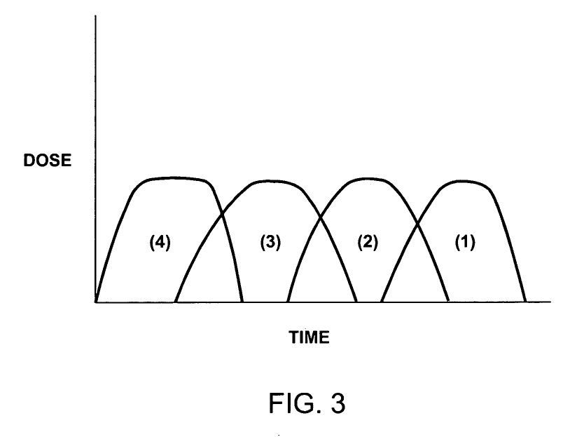

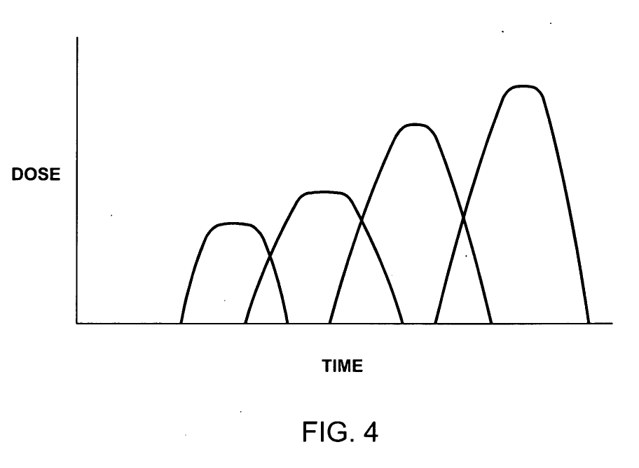

While all polymeric materials have inherent degradation rates, the degradation rates of the polymeric materials of the present invention can be altered by changing the solubility constants, molecular weights, elution profiles, and bonding strengths of the polymeric materials. The ability to vary the polymer degradation rate is advantageous because the release of pharmaceutical compounds associated with the polymer can also be controlled over time. That is, unlike prior art devices that typically release drugs immediately (see FIG. 1), the controllable drug releasing coating of the present invention allows for prolonged drug release or alternate drug elution profiles as depicted in FIGS. 3 and 4.

According to one embodiment of the present invention, those skilled in the art will appreciate that changing the solubility constant of a polymer will effect the time in which a polymer will become dissolved in a solution which also controls drug release. According to another exemplary embodiment of the present invention, the degradation rates of the polymeric materials can be controlled by altering the molecular weight of the polymers that comprise each coating layer.

The exemplary synthetic polymers of the present invention are produced by a process governed by random events. As a result, the chain lengths of individual polymer sub-units vary. Consequently, a particular polymeric material cannot be characterized by a single molecular weight. Instead, a statistical average of all of the polymeric subunits is used to denote molecular weight. The molecular weight of polymers can be expressed in different ways including number average, weight average and viscosity average. Number average is the sum of all molecular weights of the individual molecules present divided by their total number. In weight averages each polymeric subunit contributes according to the ratio of its particular molecular weight to the total.

For example, imagine a sample having five polymeric subunits of molecular weight 2, 4, 6, 8 and 10, respectively. To calculate the number average molecular weight (Mₙ), all weights of the individual polymeric subunits are added. The sum is then divided by the total number of molecules in the sample, in this case 5. Mₙ=⅖+⅘+ 6/5+ 8/5+ 10/5=6.To calculate the weight average molecular (M_{w}) weight of the above sample, the squares of each individual weight are divided by the total sum of molecular weights, in this case 30. M_{w =} 22/30+ 42/30+ 62/30+ 82/30+ 102/30=7.33. Generally speaking, weight average is more sensitive to the higher molecular weight species and number average is more sensitive to the lower molecular weight species; however, the Mₙ value will usually be within 20% of M_{w}.

With respect to viscosity average, the viscosity of a polymer solution relates to average molecular weight and can also be used to designate polymer size. Generally, polymer size is calculated by comparing the capillary efflux time (t) of a polymer dissolved in an appropriate solvent and efflux time (tₒ) for the pure solvent. Inherent polymer viscosity is then calculated by the following formula:

$$Inherent\_Viscosity \_ ( dl \text{/} g ) = \frac{[ ln ( \frac{EffluxTime\_Solution}{EffluxTime\_Solvent} ) ]}{2 \times ( Sample\_Weight \_in \_Grams}$$

Generally, lower molecular weight polymers degrade more rapidly as compared to high molecular weight polymers. In one embodiment, high molecular weight polymers are closest to the surface of the implant and low molecular weight polymers are farthest from the implant surface. Because the two layers have different molecular weights, these layers will degrade at different rates. Accordingly, any pharmaceutical compounds will also be released at different rates and at different times. Thus, the drug delivery profile of this coating can be sustained for a prolonged period of time.

Alternatively, the polymer molecular weights are varied by controlling the concentration of the monomer and activating agents. In yet another embodiment of the present invention, the molecular weight can be varied by physical means. That is, the molecular weight of the polymer chains can be reduced by cutting the chains into smaller units. For instance, the molecular weight of the polymer can be altered by exposing the polymer coating to thermal, hydrolytic, oxidative, or photo-oxidative reactions. Alternatively, the polymer molecular weight can be varied by photo-degradation or ionizing radiation such as gamma irradiation.

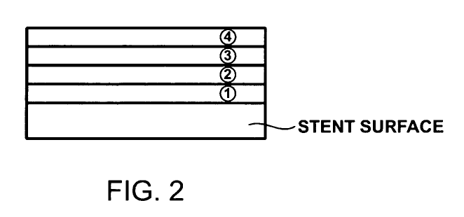

According to another exemplary embodiment of the present invention, additional layers are incorporated between the two layers to provide further variations in drug releasing profiles. The ability to add more coating layers is particularly advantageous as drug releasing can be further controlled and tailored for a desired elution profile or treatment regime. FIG. 2 illustrates an implant of the present invention having multiple coating layers applied thereon to form a polymer gradient. In this embodiment, each coating layer varies in molecular weights such that a higher molecular weight polymers are closest to the implant surface and lower weight polymers are farthest from the implant surface.

More specifically, the molecular weights of the polymer gradient coating can range, for example, from 10 kDa to 100 kDa, wherein the 100 kDa polymer layer is closest to the implant surface. In one exemplary embodiment of the present invention, the implant can comprise polymer layers having molecular weights of 100 kDa, 65 kDa, 30 kDa, and 10 kDa. Those skilled in the art will appreciate that a plurality of polymer and drug containing layers can be applied to the surface of the stent and that the preceding example was only meant to be an exemplary, but not a limiting, embodiment.

FIG. 3 illustrates the drug delivery profile of the exemplary implant illustrated in FIG. 2. Drug delivery is achieved by bulk degradation release. That is, as the polymer layer is degraded by physical stresses or chemical agents, there is complete or nearly complete release of the associated drug. Accordingly, as shown in FIG. 3, a known drug dosage can be released at particular times after the implant has been deployed in situ. For instance, as the fourth layer, which is comprised of 10 kDa polymers, is degraded, the drug (4) associated with the polymer is released. Next, once the third layer (30 kDa polymer) is degraded by the body, the associated drug (3) is released. As previously mentioned, the time differential between the degradation of the third and fourth layer is due to the different molecular weight of the polymers. This process continues until all the polymer layers and the drugs associated with the polymer layers are released from the stent As depicted in FIG. 3, drug delivery is sustained over a prolonged period of time.

According to another embodiment of the present invention, the gradient layers can include empty or blank layers. That is, not every layer needs to be provided with a pharmaceutical compound or compounds releasably bound therein. As a result, these blank layers may allow for staggered or more delayed release of a pharmaceutical compound from a subsequent layer. By providing blank layers in between gradient layers provided with pharmaceutical compounds, the present invention contemplates that drugs may be released in situ at prescribed time intervals that depend on the number of blank layers between the compound- containing layers.

In yet another embodiment of the present invention, the layers need not be limited to polymers. The controllable drug releasing coating of the present invention can also include gradients of different types of physically compatible materials. The materials that can be utilized include polyolefins, polyisobutylene and ethylene-alphaolefin copolymers; acrylic polymers and copolymers, ethylene-co-vinylacetate, polybutylmethacrylate; vinyl halide polymers and copolymers, such as polyvinyl chloride; polyvinyl ethers, such as polyvinyl methyl ether; polyvinylidene halides, such as polyvinylidene fluoride and polyvinylidene chloride; polyacrylonitrile, polyvinyl ketones; polyvinyl aromatics, such as polystyrene, polyvinyl esters, such as polyvinyl acetate; copolymers of vinyl monomers with each other and olefins, such as ethylene-methyl methacrylate copolymers, acrylonitrile-styrene copolymers, ABS resins, and ethylene-vinyl acetate copolymers; polyamides, such as Nylon 66 and polycaprolactam; polycarbonates; polyoxymethylenes; polyimides; polyethers; epoxy resins; polyurethanes; rayon; rayon-triacetate; cellulose; cellulose acetate, cellulose butyrate; cellulose acetate butyrate; cellophane; cellulose nitrate; cellulose propionate; cellulose ethers; and carboxymethyl cellulose.

As those skilled in the art will appreciate, the polymer gradient of the present invention need not be from a heavier molecular weight to a lighter molecular weight polymer. Rather, the gradient can be the converse, namely lighter molecular weight to heavier weight polymers. Because a higher molecular weight coating layer is the outermost layer in such an embodiment, drug delivery is delayed as more time is required to physically or chemically degrade this higher molecular weight layer after the implant has been delivered in situ. Additionally, those skilled in the art will appreciate that the molecular weights of the coating layers can be varied such that the polymer gradient is non-linear. That is, unlike the previous embodiments, the molecular weights of the individual coating layers can vary in weight without a linearly distinct pattern. Consequently, unlike prior art polymer coatings, the gradient coatings of the present invention allow for the controllable release of drugs by altering the molecular weights or other properties of the individual layers over a wide range of linear and non-linear gradients. This controllable release of drugs is advantageous as conditions and/or diseases having delayed pathologies may be more effectively treated at the proper time.

Additionally, controlling the release of drugs from the polymer gradient may be achieved by adjusting other physical properties of the layers such as binding strengths between the polymers and the drugs. That is, the ease or rate that the drug is released from the polymer can be affected by the strength of the bond between the drug and the polymer. That is, a stronger bond (e.g., covalent bond) is more difficult to break as compared to a weaker bond (e.g., ionic, polar). Thus, a stronger bond will take longer to break as compared to a weaker bond as more energy would be required to release the drug from the stronger bond polymer. Consequently, depending on the strength of the bond between the drug and the polymer, the time at which the drug is released from the coating can be controlled within the teachings of the present invention.

Alternatively, the present invention also contemplates that the pharmaceutical compounds need not be bound to the individual gradient layers. Rather, the pharmaceutical compounds can be sealed between adjacent coating layers. The entrapped compounds are then released as the individual coating layers are degraded. In another embodiment, the entrapped compounds can diffuse through the polymer layer. That is, the polymer layer is porous which allows the entrapped compounds to be released from the polymer. According to yet another embodiment of the present invention, pharmaceutical compounds can be incorporated into the polymer coating by imbibing the compounds into the polymer coating with an organic solvent. That is, the polymer layer is treated so that it will swell thereby allowing for the absorption of the pharmaceutical compounds by the polymer coating.

In addition to various methods of releasing one or more associated pharmaceutical compounds from the gradient polymers, the present invention contemplates that the amount of drugs that are released from polymers can also be altered. While each polymer layer is generally provided with the same or nearly the same amount of drugs as shown in FIG. 3, the individual layers of the gradient coating may incorporate more or less drug than an adjacent layer. For example, FIG. 4 illustrates an implant wherein the greatest drug dosage is contained in the coating layer closest to the implant surface and the farthest layer from the implant surface contains the lowest drug dosage. Thus, the gradient coatings of the present invention are capable of releasing one or more drugs in different quantities and at different times through a variety of mechanisms.

The pharmaceutical compounds that can be released by the gradient coatings of the present invention may be anti-restenotic or anti-thrombogenic compounds. Exemplary compounds include, without limitation, angiopeptin (a somatostatin analog), calcium channel blockers, angiotensin converting enzyme inhibitors (captopril, cilazapril), cyclosporin A, trapidil (an antianginal, antiplatelet agent), terbinafine (antifungal), colchicine and taxol (antitubulin antproliferabves), c-myc and c-myb antinsense oligonucleotides, and heparin.

It is also contemplated as being within the scope of the present invention that the gradient coatings can include, without limitation, antibacterial agents, antiparasitic agents, antiviral agents, antifungal agents, amoebicidal agents, trichomonacidal agents, protease inhibitors, antihistamines, anti-inflammatory agents, anticholinergic agents, immunoglobulins, antigens, ophthalmic agents, chelating agents, immunosuppressive agents, antimetabolites, anesthetics, analgesic agents, antiarthritic agents, antiasthmatic agents, anticoagulants, antithrombogenic agents, anticonvulsants, antidepressants, antidiabetic agents, antineoplastics, antipsychotic agents, antihypertensive agents, muscle relaxants, proteins, peptides, hormones and lubricating agents.

In one embodiment of the present invention, the gradient coating can also include macrolide antibiotics such as rapamycin and analogues and derivatives thereof such as, but not limited to, those described in U.S. Pat. Nos. 5,665,772, 5,258,389, 6,015,815, and 6,329,386. The disclosures of the aforementioned United States Patents are hereby incorporated by reference in their entirety.

The controllable releasing gradient coatings of the present invention can be applied to a wide variety of implants including, but not limited to, stents, catheters, micro-particles, probes, vascular grafts, access devices, in-dwelling access ports, valves, plates, barriers, supports, shunts, discs, and joints, as well as virtually any device intended to spend time within a patient's body or vasculature. More specifically, the coatings of the present invention can be applied to stents such as, but not limited to, vascular stents, biliary stents, and esophogeal stents. Applying the gradient coatings of the present invention to stents is particularly advantageous because stents provide mechanical support to maintain the patency or openness of a vessel or hollow organ while controllably releasing an effective drug dose to the site of implantation over prolonged periods of time.

According to the teachings of the present invention, it is also contemplated that the controllable releasing gradient coatings can be applied to metallic materials such as, but not limited to, aluminum, 316L stainless steel, MP35N alloy, superelastic Nitinol nickel-titanium, titanium alloys, and other alloys such as a wrought Cobalt-Chromium-Nickel-Molybdenum-Iron alloy. Furthermore, the gradient coatings can be applied to bioresorbable polymers such as, but not limited to, polyanhydrides, polycaprolactones, polyglycolic acids, poly-L-lactic acids, polydioxanone, polyphosphate esters, or blends thereof, such as poly-D-L-lactic acids.

Depending upon the type of materials used to form the gradient coatings of the present invention, the coatings can be applied to the surface of a medical device through any of the coating processes known or developed in the art. One method includes directly bonding the gradient coating to the implant's surface. By directly attaching the polymer coating to the implant, covalent chemical bonding techniques are utilized. Generally, the implant surface possesses chemical functional groups on its surface such as carbonyl groups, primary amines, hydroxyl groups, or silane groups which will form strong, chemical bonds with similar groups on the active compounds utilized. In the absence of such chemical forming functional group, known techniques can be utilized to activate the material's surface before coupling the biological compound. Surface activation is a process of generating, or producing, reactive chemical functional groups using chemical or physical techniques such as, but not limited to, ionization, heating, photochemical activation, oxidizing acids, and etching with strong organic solvents.

Alternatively, the gradient coating can be indirectly bound to the implant's surface through an intermediate layer (not shown). This intermediate layer can be either covalently bound to the fixed substrate's surface or bonded through intermolecular attractions such as ionic or Van der Waals forces. Examples of commonly used intermediate layers within the scope of the present invention include, but are not limited to, organic polymers such as silicones, polyamines, polystyrene, polyurethane, acrylates, methoxysilanes, and others.

According to the teachings of the present invention, the implant also can be provided with a non-erodible base coating. The base coating can be provided so as to enhance the biocompatibility of the implant. Exemplary base coatings can be selected from the group consisting of polyurethanes, silicones and polysilanes. Other polymers that can be utilized include polyolefins, polyisobutylene and ethylene-alphaolefin copolymers; acrylic polymers and copolymers, ethylene-co- vinylacetate, polybutylmethacrylate; vinyl halide polymers and copolymers, such as polyvinyl chloride; polyvinyl ethers, such as polyvinyl methyl ether, polyvinylidene halides, such as polyvinylidene fluoride and polyvinylidene chloride; polyacrylonitrile, polyvinyl ketones; polyvinyl aromatics, such as polystyrene, polyvinyl esters, such as polyvinyl acetate; copolymers of vinyl monomers with each other and olefins, such as ethylene-methyl methacrylate copolymers, acrylonitrile-styrene copolymers, ABS resins, and ethylene-vinyl acetate copolymers; polyamides, such as Nylon 66 and polycaprolactam; polycarbonates; polyoxymethylenes; polyimides; polyethers; epoxy resins; polyurethanes; rayon; rayon-triacetate; cellulose; cellulose acetate, cellulose butyrate; cellulose acetate butyrate; cellophane; cellulose nitrate; cellulose propionate; cellulose ethers; and carboxymethyl cellulose. In accordance with the teachings of the present invention, the base coating can also include, without limitation, antibiotics, anti-inflammatory agents, lubricity-enhancing agents, anti- coagulants, anti-metabolites, anti-thrombogenic agents, immunosuppressive agents, muscle relaxants, proteins, peptides, and hormones.

Pharmaceutical compounds can be applied to the implant surfaces by various methods according to the teachings of the present invention. One exemplary method includes adding the pharmaceutical compounds to the solvated polymer to form a drug/polymer solution. The drug/polymer solution can then be applied directly to the surface of the implant; for example, by either spraying or dip coating the implant. As the solvent dries or evaporates, the polymer/drug coating is deposited on the implant. Furthermore, multiple applications can be used to ensure that the coating is generally uniform and a sufficient amount of the drug has been applied to the implant surface.

In use, the implant of the present invention having controllable drug releasing gradient coatings are delivered to a target site by any processes known or developed in the art. For instance, a gradient coated vascular stent can be delivered to the vasculature via a ballon catheter. Once implanted, the gradient coating is exposed to both physical stresses and to chemical agents within the body such as flowing blood and various enzymes and proteins found in the blood. Depending on the solubility, molecular weight, binding strengths, and other physical properties of the gradient coatings, any pharmaceutical compounds associated with the individual coating layers can be released from the implant according to a desired drug elution profile designed in accordance with the teachings of the present invention.

Unless otherwise indicated, all numbers expressing quantities of ingredients, properties such as molecular weight, reaction conditions, and so forth used in the specification and claims are to be understood as being modified in all instances by the term “about” Accordingly, unless indicated to the contrary, the numerical parameters set forth in the foregoing specification and attached claims are approximations that can vary depending upon the desired properties sought to be obtained by those skilled in the art utilizing the teachings of the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviations found in their respective testing measurements.

In closing, it is to be understood that the embodiments of the present invention disclosed herein are illustrative of the principles of the present invention. Other modifications that can be employed that are within the scope of the invention. Thus, by way of example, but not of limitation, alterative configurations of the present invention can be utilized in accordance with the teachings herein. Accordingly, the present invention is not limited to that precisely shown and described.

What is claimed:

1. A medical implant for the controllable delivery of at least one pharmaceutical compound to a localized area within a patient, said implant comprising:
an implantable medical device having a surface and a coating formed on at least a portion of said surface, said coating having at least two layers, at least one of said layers incorporating at least one releasable pharmaceutical compound, each of said layers having at least one physical property affecting the releasability of said releasable pharmaceutical compound that differs from said at least one other layer.

2. The medical implant of claim 1 wherein said medical device is selected from the group consisting of stents, probes, catheters, micro-particles, pacing leads, vascular grafts, access devices, in-dwelling access ports, valves, plates, barriers, supports, shunts, discs, and joints.

3. The medical implant of claim 2 wherein said stent is selected from the group consisting of vascular stents, biliary stents, and esophogeal stents.

4. The medical implant of claim 1 wherein said at least one layer is a polymer.

5. The medical implant of claim 4 wherein said at least one physical property affecting the releasability of said at least one pharmaceutical compound is molecular weight.

6. The medical implant of claim 5 wherein said molecular weight range from about 1 kDa to 100,000 kDa.

7. The medical implant of claim 4 wherein said polymer is selected from the group consisting of poly(caprolactone), poly(lactic acid), poly(glycolic acid), poly(ethylene-vinyl acetate), collagen, heparinized collagen, polyvinyl pyrrolidone, polytetrafluoroethylene, polyethylene glycol, polystyrene, acrylates, polyesters, epoxides, silicones, cellulose, and copolymers thereof.

8. The medical implant of claim 1 wherein said at least one pharmaceutical compound is an anti-restentoic drug.

9. The medical implant of claim 8 wherein said anti-restenotic compound is a macrolide antibiotic.

10. The medical implant of claim 9 wherein the macrolide antibiotic is rapamycin or analogues and derivatives thereof.

11. A method for controllably delivering at least one pharmaceutical compound to a localized area within a patient, said method comprising the steps of:
providing a controllable drug releasing gradient coating on an implantable medical device; and
implanting said medical device at a specific target site within a patient.

12. A method for making a controllable drug releasing gradient coating for the surface of a medical device, said method comprising the steps of:
forming a first layer on said surface of said medical device, said first layer containing at least one releasably bound pharmaceutical compound and having at least one physical property affecting the releasability of said at least one pharmaceutical compound; and
forming at least one additional layer on said first layer, said at least one additional layer differing in said at least one physical property.

13. The method of claim 12 wherein said generally tubular structure is a stent or a catheter.

14. The method of claim 13 wherein said stent is self-expanding.

15. The method of claim 13 wherein said stent is mechanically expandable.

16. The method of claim 13 wherein said stent is bioresorbable.

17. The method of claim 12 wherein each polymer layer of said at least one polymer layer is comprised of polymers having different molecular weights.

18. The method of claim 17 wherein said molecular weights range from about 1 kDa to 100,000 kDa.

19. The method of claim 12 wherein said polymer layers are selected from the group consisting of poly(caprolactone), poly(lactic acid), poly(glycolic acid), poly(ethylene-vinyl acetate), collagen, heparinized collagen, polyvinyl pyrrolidone, polytetrafluoroethylene, polyethylene glycol, polystyrene, acrylates, polyesters, epoxides, silicones, cellulose, and copolymers thereof.

20. The method of claim 17 wherein said at least one anti-restenotic compound is contained within adjacent polymer coatings.

21. The method of claim 20 wherein said anti-restenotic compound is a macrolide antibiotic.

22. The method of claim 21 wherein the macrolide antibiotic is rapamycin or analogues and derivatives thereof.

23. The method of claim 17 wherein said at least one anti-restenotic compound is coupled to said polymer coating.

24. The method of claim 23 wherein said anti-restenotic compound is a macrolide antibiotic.

25. The method of claim 24 wherein the macrolide antibiotic is rapamycin or analogues and derivatives thereof.
